(19) United States
(12) Patent Application Publication
(10) Pub. No.: US 2017/0157081 A1
(43) Pub. Date: Jun. 8, 2017

(54) SILAGE COMPOSITIONS AND METHODS OF MAKING AND USING THE SAME

(71) Applicant: BENEMILK OY, Raisio (FI)

(72) Inventors: Illimar ALTOSAAR, Ottawa (CA); Ilmo Pellervo ARONEN, Hinnerjoki (FI); Craig Cano BEESON, Charleston, SC (US); Christopher John BUNTEL, Singapore (SG)

(73) Assignee: BENEMILK OY, Raisio (FI)

(21) Appl. No.: 15/328,021

(22) PCT Filed: Jul. 21, 2014
(86) PCT No.: PCT/US14/47472

§ 371 Date: Jan. 21, 2017

(51) Int. Cl. A61K 31/20; A23K 10/30; A61K 36/48; A23K 50/10; A61K 36/899; A23K 30/18; A23K 20/158

(52) U.S. Cl. CPC A61K 31/20; A23K 30/18; A23K 10/30; A23K 20/158; A23K 50/10; A61K 36/899; A61K 36/48

(57) ABSTRACT

Silage compositions are described herein, as well as methods for their preparation and use. A silage composition may include a fatty acid component comprising at least about 70% saturated fatty acid by weight and a fermented component. The fatty acid component may be present in the silage composition in an amount of at least about 10% by weight of the silage composition.

### BACKGROUND

Increasing production and fat content of milk obtained from lactating ruminants has been a major goal for dairy farmers. Additional milk production per ruminant is beneficial because it results in a higher yield, thereby increasing profits. Increased milk fat is desirable because it has a higher economic value than skimmed milk itself and can be used in highly desirable food products, such as butter, cheese, yogurt, and the like.

A common approach to increasing either or both of production and milk fat content includes adjusting feed, nutrients, elements, vitamins, supplements, and/or the like provided to the ruminant. One such specific method includes feeding the ruminant a total mixed ration (TMR), which is a mix of grain and silage with some protein meals, such as, for example, soya bean meal and canola meal. Additional materials and trace elements, vitamins, extra nutrients, and the like may also be added to the TMR.

However, the current methods and feeds used to increase milk fat content tend to lower milk production, lower protein content, and/or have other detrimental effects on the ruminant and on farmers' outcomes. Furthermore, the methods and feeds often result in other undesired effects, such as increased trans fatty acid levels on the fatty acid profile of the milk fat.

### SUMMARY

In an embodiment, a silage composition may include a fatty acid component having at least about 70% saturated fatty acid by weight and a fermented component. The fatty acid component may be present in the silage composition in an amount of at least about 10% by weight of the silage composition.

In an embodiment, a method of preparing a silage composition for ruminants may include combining a first component having a fatty acid composition and a second component to form a mixture such that the fatty acid is present in the mixture in an amount of at least about 10% by weight of the mixture. The method may further include allowing the mixture to ferment.

In an embodiment, a method of increasing milk fat content in ruminants may include providing a silage composition to a ruminant for ingestion. The silage composition may include a fatty acid component having at least about 70% saturated fatty acid by weight and a fermented component. The fatty acid component may be present in the silage composition in an amount of at least about 10% by weight of the silage composition.

In an embodiment, a silage composition may include a fatty acid component having at least about 70% saturated fatty acid by weight and a fermented component. The fatty acid component may be present in the silage composition in an amount of less than about 10% by weight of the silage composition.

In an embodiment, a method of preparing a silage composition for ruminants may include combining a first component having a fatty acid composition and a second component to form a mixture such that the fatty acid is present in the mixture in an amount of less than about 10% by weight of the mixture. The method may further include allowing the mixture to ferment.

In an embodiment, a method of increasing milk fat content in ruminants may include providing a silage composition to a ruminant for ingestion. The silage composition may include a fatty acid component having at least about 70% saturated fatty acid by weight and a fermented component. The fatty acid component may be present in the silage composition in an amount of less than about 10% by weight of the silage composition.

### BRIEF DESCRIPTION OF THE DRAWINGS

FIG. 1 depicts a flow diagram of a method of preparing a silage composition according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The following terms shall have, for the purposes of this application, the respective meanings set forth below.

A ruminant is a class of mammal with a multiple chamber stomach that gives the animal an ability to digest cellulose-based food. The stomach of a ruminant has four morphologically distinct compartments: the rumen, the reticulum, the omasum, and the abomasum. Bacteria in the rumen enable the ruminant to digest cellulose-based food by softening it and regurgitating the semi-digested mass. The regurgitate, known as cud, is then chewed again by the ruminant. Specific examples of ruminants include, but are not limited to, cattle, bison, buffaloes, yaks, camels, llamas, giraffes, deer, pronghorns, antelopes, sheep, and goats. The milk produced by ruminants is widely used in a variety of dairy-based products. For example, dairy cows are of considerable commercial significance for the production of milk and processed dairy products such as, for example, yogurt, cheese, whey, and ice cream.

A silage composition is generally a fermented, high-moisture fodder that can be fed to ruminants. The silage composition can be fermented and stored as part of a silaging process, which generally includes placing various ingredients into a silo or another structure or container configured to exclude air. Optionally, the ingredients can be wrapped in a polymer sheet or film. The ingredients are allowed to ferment in the structure or container, thereby retarding spoilage. Silage frequently can have a water content of about 60% to about 80% by weight.

The present disclosure relates generally to silage compositions that can be prepared via a typical silaging process and fed to ruminants for purposes of affecting milk production in the ruminant. Particularly, the silage compositions described herein may be fed to a ruminant to increase the amount of milk produced by the ruminant and/or to increase the fat content of the milk produced by the ruminant, as described in greater detail herein.

When a ruminant consumes feed, the fat in the feed is modified by the rumen to provide a milk fat profile that is different from the profile of fat in the feed. All fats which are not completely inert in the rumen may decrease rumen digestibility of the feed material. Milk composition and fat quality can be influenced by the ruminant's diet. For example, oil feeding can have negative effects on both rumen function and milk formation. As a result of oil feeding, milk protein concentration is lowered, fat concentration is decreased, and proportion of trans fatty acids is increased in ruminants. These have been connected especially to an increase in harmful low-density lipoprotein (LDL) cholesterol and to a decrease in beneficial high-density lipoprotein (HDL) cholesterol in human blood when the milk is consumed. In addition, the properties of the milk fat during industrial milk processing are weakened. A high level of polyunsaturated fatty acids in milk can also cause taste defects and preservation problems. A typical fatty acid composition of milk fat may contain more than 70% saturated fatty acids, and the total amount of trans fatty acids may vary in the range of 3%-10%. When vegetable oil is added into the feed, the proportion of trans fatty acids may rise to more than 10%.

One solution to diminishing the detrimental effect of oil and fat is to prevent triglyceride fat hydrolysis. Fat hydrolysis can be decreased, for example, by protecting fats with formaldehyde treated casein. Another alternative is to make insoluble fatty acid calcium salts whereby hydrogenation in rumen can be avoided. However, fatty acid salts have a pungent taste, which can limit their acceptability and usability in feeds and can result in decreased feed intake. The salts may also impact the ability to pelletize the feed.

Accordingly, the silage compositions described herein may allow for a transfer of palmitic acid from the composition via the digestive tract into the blood circulation of a ruminant, which may improve the energy efficiency of milk production of the ruminant. When the utilization of energy becomes more efficient, milk production increases and the concentrations of protein and fat in the milk rise. Especially, the silage composition enhances fat synthesis in the mammary gland by bringing milk fat components to the cell. As a result, the energy-consuming synthesis in the mammary gland may not be necessary. Thus, glucose may be more efficiently used for lactose production, whereupon milk production increases. The milk protein content rises because glucose need not be produced from amino acids. Thus, the ruminant may not lose as much weight at the beginning of the lactation period.

In the various embodiments described herein, the silage compositions may include at least a fatty acid component and a fermented component. The fatty acid component may be primarily one or more saturated fatty acids (such as palmitic acid) and may contain little or no unsaturated trans fatty acids, as described in greater detail herein. The fatty acid component may be present in the silage composition in an amount of at least about 10% by weight of the silage composition, including, for example, about 10% to about 50%, about 10% to about 60%, about 10% to about 80%, or about 10% to about 90% by weight of the silage composition. Specific examples of amounts by weight of the silage composition include about 10%, about 20%, about 30%, about 40%, about 50%, about 60%, about 70%, about 80%, about 90%, or any value or range between any two of these values (including endpoints).

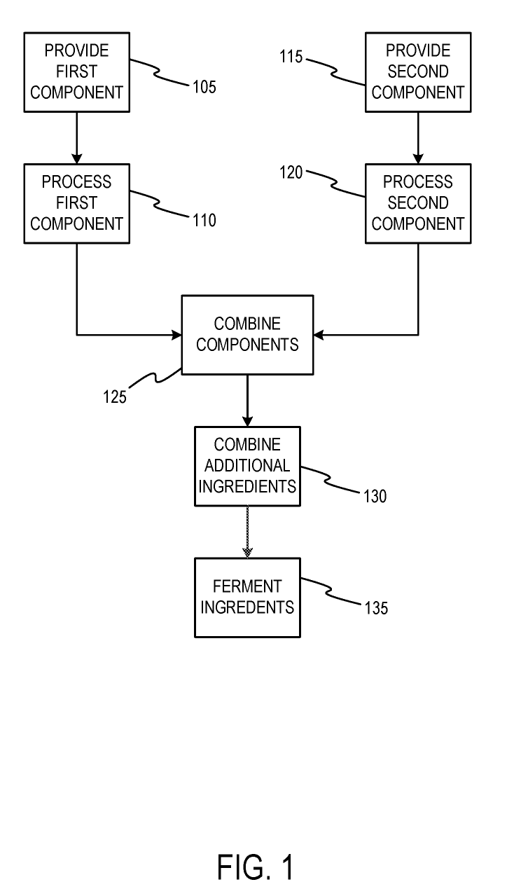

The silage compositions described herein, once prepared (as described herein with respect to FIG. 1), may generally include at least a fatty acid component and a fermented component. In some embodiments, the fatty acid component may be at least about 70% saturated fatty acid by weight. Thus, the fatty acid component may contain saturated fatty acid in an amount of about 70% by weight, about 80% by weight, about 90% by weight, about 95% by weight, about 96% by weight, about 97% by weight, about 98% by weight, about 99% by weight, about 100% by weight, or any value or range between any two of these values (including endpoints).

In some embodiments, the silage composition may consist essentially of the fatty acid component and the fermented component. In other embodiments, the silage composition may consist of the fatty acid component and the fermented component. In other embodiments, the silage composition may include other components in addition to the fatty acid component and the fermented component, as described in greater detail herein.

In various embodiments, the fatty acid component may generally include one or more free fatty acids and/or glycolipids. Free fatty acids may generally be unconjugated fatty acids, whereas glycolipids may be fatty acids conjugated with a carbohydrate. In some embodiments, the fatty acid component may be present in the silage composition in an amount of at least about 10% by weight of the silage composition, including, for example, about 30% by weight or about 50% by weight of the silage composition. In particular embodiments, the fatty acid component may be present in the silage composition in an amount of about 10% by weight, about 15% by weight, about 20% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, or any value or range between any two of these values. In some embodiments, the fatty acid component may represent about 30% to about 50%, about 30% to about 90%, or about 40% to about 60% by weight of the silage composition.

In other embodiments, the fatty acid component may be present in the silage composition in an amount that is less than about 10% by weight of the silage composition. Thus, for example, the fatty acid component may be present in the silage composition in an amount of about 0.1% by weight to about 10% by weight of the silage composition, including about 0.1% by weight, about 0.5% by weight, about 1% by weight, about 2% by weight, about 3% by weight, about 4% by weight, about 5% by weight, about 6% by weight, about 7% by weight, about 8% by weight, about 9% by weight, about 10% by weight, or any value or range between any two of these values (including endpoints). Accordingly, in such embodiments, for every 1000 kg of silage composition, about 1 kg to about 100 kg of the fatty acid component may be included in the silage composition, including about 1 kg, about 5 kg, about 10 kg, about 20 kg, about 30 kg, about 40 kg, about 50 kg, about 60 kg, about 70 kg, about 80 kg, about 90 kg, about 100 kg, or any value or range between any two of these values (including endpoints).

In some embodiments, the fatty acid component may have a melting point equal to or greater than about 40° C. In some embodiments, the fatty acid component may have a melting point equal to or less than about 80° C. In some embodiments, the fatty acid component may have a melting point of about 40° C. to about 80° C. In some embodiments, the fatty acid component may have a melting point of about 60° C. to about 80° C. In some embodiments, the fatty acid component may have a melting point of about 63° C. to about 65° C. In particular embodiments, the fatty acid component may have a melting point of about 40° C., about 45° C., about 50° C., about 55° C., about 60° C., about 65° C., about 70° C., about 75° C., about 80° C., or any value or range between any two of these values (including endpoints). The melting point may generally be selected so that it is a temperature that ensures that the fatty acid is inert in the rumen environment.

As previously described herein, the fatty acid component may include at least one saturated fatty acid. For example, the fatty acid component may include 1, 2, 3, 4, 5, 6, or more different saturated fatty acids. In some embodiments, the saturated fatty acid may be present in the fatty acid component in an amount that results in a ruminant consuming the silage composition to produce a desired quality and quantity of milk, as described in greater detail herein. Thus, in some embodiments, the saturated fatty acid may be present in an amount of at least about 70% by weight of the fatty acid component. In particular embodiments, the saturated fatty acid may be present in an amount of about 70% by weight of the fatty acid component to about 100% by weight of the fatty acid component, including about 70% by weight, about 71% by weight, about 72% by weight, about 73% by weight, about 74% by weight, about 75% by weight, about 76% by weight, about 77% by weight, about 78% by weight, about 79% by weight, about 80% by weight, about 81% by weight, about 82% by weight, about 83% by weight, about 84% by weight, about 85% by weight, about 86% by weight, about 87% by weight, about 88% by weight, about 89% by weight, about 90% by weight, about 91% by weight, about 92% by weight, about 93% by weight, about 94% by weight, about 95% by weight, about 96% by weight, about 97% by weight, about 98% by weight, about 99% by weight, about 100% by weight, or any value or range between any two of these values (including endpoints). The saturated fatty acid is not limited by this disclosure, and may include any number of saturated fatty acids now known or later discovered, including all derivatives thereof. For example, derivatives of a saturated fatty acid may include salts, esters, amides, carbonates, carbamates, imides, anhydrides, alcohols of a fatty acid, and/or the like.

As used herein, a salt of the fatty acid may be any acid addition salt, including, but not limited to, halogenic acid salts such as, for example, hydrobromic, hydrochloric, hydrofluoric, and hydroiodic acid salts; inorganic acid salts such as, for example, nitric, perchloric, sulfuric, and phosphoric acid salts; organic acid salts such as, for example, sulfonic acid salts (methanesulfonic, trifluoromethane sulfonic, ethanesulfonic, benzenesulfonic, or p-toluenesulfonic), acetic, malic, fumaric, succinic, citric, benzoic, gluconic, lactic, mandelic, mucic, pamoic, pantothenic, oxalic, and maleic acid salts; and amino acid salts such as aspartic or glutamic acid salts. The acid addition salt may be a mono- or di-acid addition salt, such as a di-hydrohalogenic, di-sulfuric, di-phosphoric, or di-organic acid salt. In all cases, the acid addition salt is used as an achiral reagent which is not selected on the basis of any expected or known preference for interaction with or precipitation of a specific optical isomer of the products of this disclosure.

A fatty acid ester, as used herein, means an ester of a fatty acid. For example, the fatty acid ester may be in a form of RCOOR′. R may be any saturated or unsaturated alkyl group including, without limitation, C10, C12, C14, C16, C18, C20, C22 and C24. R′ may be any groups having from about 1 to about 1000 carbon atoms and with or without hetero atoms. In some embodiments, R′ may have from about 1 to about 20, from about 3 to about 10, or from about 5 to about 15 carbon atoms. The hetero atoms may include, without limitation, N, O, S, P, Se, halogen, Si, and B. For example, R′ may be a C₁₋₆alkyl, such as methyl, ethyl or t-butyl; a C₁₋₆alkoxyC₁₋₆alkyl; a heterocyclyl, such as tetrahydrofuranyl; a C₆₋₁₀aryloxyC₁₋₆alkyl, such as benzyloxymethyl (BOM); a silyl, such as trimethylsilyl, t-butyldimethylsilyl and t-butyldiphenylsilyl; a cinnamyl; an allyl; a C₁₋₆alkyl which is mono-, di- or trisubstituted by halogen, silyl, cyano or C₁₋₆aryl, wherein the aryl ring is unsubstituted or substituted by one, two, or three residues selected from the group consisting of C₁₋₇alkyl, C₁₋₇alkoxy, halogen, nitro, cyano and CF₃; or a C₁₋₂alkyl substituted by 9-fluorenyl.

As used herein, a fatty acid amide may generally include amides of fatty acids where the fatty acid is bonded to an amide group. For example, the fatty acid amide may have a formula of RCONR′R″. R may be any saturated or unsaturated alkyl group including, without limitation, C10, C12, C14, C16, C18, C20, C22, and C24. R′ and R″ may each be any group having from about 1 to about 1000 carbon atoms and with or without hetero atoms. In some embodiments, R′ may have from about 1 to about 20, from about 3 to about 10, or from about 5 to about 15 carbon atoms. The hetero atoms may include, without limitation, N, O, S, P, Se, halogen, Si, and B. For example, R′ and R″ each may be an alkyl, an alkenyl, an alkynyl, an aryl, an aralkyl, a cycloalkyl, a halogenated alkyl, or a heterocycloalkyl group.

A fatty acid anhydride may generally refer to a compound which results from the condensation of a fatty acid with a carboxylic acid. Illustrative examples of carboxylic acids that may be used to form a fatty acid anhydride include acetic acid, propionic acid, benzoic acid, and the like.

An alcohol of a fatty acid refers to a fatty acid having a straight chain or branched, saturated, radical groups. The fatty acid alcohol may additionally have 3-30 carbon atoms and one or more hydroxy groups. The alkyl portion of the alcohol component can be propyl, butyl, pentyl, hexyl, iso-propyl, iso-butyl, sec-butyl, tert-butyl, or the like. One of skill in the art may appreciate that other alcohol groups may also be useful in the present disclosure.

In some embodiments, the saturated fatty acid may include a palmitic acid compound. The palmitic acid compound is not limited by this disclosure, and may include one or more of a conjugated palmitic acid, unconjugated palmitic acid, free palmitic acid, palmitic acid derivatives, and/or the like. Palmitic acid, also known as hexadecanoic acid, has a molecular formula of CH₃(CH₂)₁₄CO₂H. Specific examples of palmitic acid derivatives may include palmitic acid esters, palmitic acid amides, palmitic acid salts, palmitic acid carbonates, palmitic acid carbamates, palmitic acid imides, palmitic acid anhydrides, and/or the like. The palmitic acid compound may be present in the fatty acid component in an amount of at least about 60% by weight of the fatty acid component, such as, for example, about 60% by weight of the fatty acid to about 100% by weight of the fatty acid, including about 60% by weight, about 65% by weight, about 70% by weight, about 75% by weight, about 80% by weight, about 85% by weight, about 90% by weight, about 95% by weight, about 98% by weight, about 99% by weight, about 100% by weight, or any value or range between any two of these values (including endpoints). In some embodiments, the palmitic acid compound may be present in the fatty acid component in an amount of at least about 70% by weight of the fatty acid component. In some embodiments, the palmitic acid compound may be present in the fatty acid component in an amount of at least about 80% by weight of the fatty acid component. In some embodiments, the palmitic acid compound may be present in the fatty acid component in an amount of at least about 90% by weight of the fatty acid component. In some embodiments, the fatty acid component may consist essentially of the palmitic acid compound. In other embodiments, the fatty acid component may consist of or be entirely composed of the palmitic acid compound.

In some embodiments, the fatty acid component may include an unsaturated fatty acid. Unsaturated fatty acid, as used herein, refers to any mono- or polyunsaturated fat, and includes unsaturated trans fatty acids. The unsaturated fatty acids must contain at least one alkene bond and may contain two or more alkene groups in any position in the hydrocarbon chain, and the unsaturation may or may not be present as a conjugated system of double bonds. The unsaturated fatty acid is not limited by this disclosure, and may include any number of unsaturated fatty acids now known or later discovered, including all derivatives thereof. For example, derivatives of an unsaturated fatty acid may include salts, esters, amides, anhydrides, alcohols, and/or the like, as previously described herein. In various embodiments, an amount of unsaturated fatty acid may be used in the fatty acid component to affect a desired quality of milk produced by the ruminant consuming the silage composition, as described in greater detail herein. Thus, in some embodiments, the fatty acid component may be substantially free of unsaturated fatty acids. As used herein with respect to unsaturated fatty acids, the term “substantially free” is understood to mean substantially no amount of unsaturated fatty acids or about 10% or less by weight of unsaturated fatty acids, including trace amounts of unsaturated fatty acids. Accordingly, the unsaturated fatty acid may be present in the fatty acid component in an amount of about 10% or less by weight of the fatty acid component, including about 10% or less by weight, about 5% or less by weight, about 4% or less by weight, about 3% or less by weight, about 2% or less by weight, about 1% or less by weight, about 0.5% or less by weight, about 0% by weight, or any value or range between any two of these values.

The fermented component may generally be any component that is capable of fermenting when it is placed into a container during a silaging process. In some embodiments, the fermented component may be a green plant. In some embodiments, the fermented component may include at least one of a grass, a weed, a clover, alfalfa, a vetch, straw, oat, rye, sorghum, a cereal grain, hay, and maize. Those having ordinary skill in the art will recognize other components that are capable of fermenting that may also be used within the scope of this disclosure.

Various silage compositions may also have at least one additional ingredient. Illustrative examples of additional ingredients include, but are not limited to, an additive, urea, anhydrous ammonia, an acid, a mineral, an enzyme, and the like, or a combination thereof. The various silage compositions may include the additional ingredients in various amounts necessary to provide beneficial nutritional and dietary needs of the ruminant that is to consume the silage composition. For example, other ingredients may include at least one acid and/or at least one mineral, each in an amount sufficient to provide beneficial nutritional and dietary needs of the ruminant.

In some embodiments, the acid may be at least one of formic acid, a mineral acid, propionic acid, lactic acid, or sodium diacetate. Formic acid may be used, for example, as a preservative and/or an antibacterial agent in the silage composition. In addition, formic acid may be used to promote fermentation of lactic acid and to reduce or suppress the formation of butyric acid. In some embodiments, formic acid may allow fermentation to occur quicker than fermentation of a silage composition not containing formic acid. In some embodiments, formic acid may allow fermentation to occur at a lower temperature relative to a silage composition not containing formic acid. In some embodiments, formic acid may function to preserve various nutrients present in the silage composition. In some embodiments, formic acid may be converted to lactic acid during the fermentation process. In some embodiments, formic acid may not be required, as lactic acid may be formed naturally during the fermentation process.

The mineral acid may include, for example, sulfuric acid and/or hydrochloric acid. A mineral acid may be used, for example, as a general purpose food additive to produce various food acids such as citric acid and lactic acid, or to directly control the pH of the silage composition. In some embodiments, propionic acid may be used as a preservative and/or to inhibit or prevent growth of mold and/or bacteria on the silage composition. Lactic acid may be used, for example, as a preservative, a curing agent, and/or a flavoring agent. Sodium diacetate may be used, for example, as a preservative and/or a flavoring agent.

The mineral may be any mineral that is a generally recognized as safe (GRAS) mineral or a combination of such minerals. The mineral may further be obtained from any mineral source that provides a bioavailable mineral. In some embodiments, the mineral may be one or more of calcium, sodium, magnesium, potassium, phosphorous, zinc, selenium, manganese, iron, cobalt, copper, iodine, molybdenum, and/or the like, including ions of any of the foregoing. In some embodiments, the mineral may be selected from one or more of a sodium salt, a calcium salt, a magnesium salt, a cobalt salt, a manganese salt, a potassium salt, an iron salt, a zinc salt, copper sulfate, copper oxide, selenium yeast, a chelated mineral, and/or the like. Illustrative examples of sodium salts include monosodium phosphate, sodium acetate, sodium chloride, sodium bicarbonate, disodium phosphate, sodium iodate, sodium iodide, sodium tripolyphosphate, sodium sulfate, sodium selenite, and/or the like. Illustrative examples of calcium salts include calcium acetate, calcium carbonate, calcium chloride, calcium gluconate, calcium hydroxide, calcium iodate, calcium iodobehenate, calcium oxide, anhydrous calcium sulfate, calcium sulfate dehydrate, dicalcium phosphate, monocalcium phosphate, tricalcium phosphate, and/or the like. Illustrative magnesium salts include magnesium acetate, magnesium carbonate, magnesium oxide, magnesium sulfate, and/or the like. Illustrative cobalt salts include cobalt acetate, cobalt carbonate, cobalt chloride, cobalt oxide, cobalt sulfate, and/or the like. Illustrative examples of manganese salts include manganese carbonate, manganese chloride, manganese citrate, manganese gluconate, manganese orthophosphate, manganese oxide, manganese phosphate, manganese sulfate, and/or the like. Illustrative examples of potassium salts include potassium acetate, potassium bicarbonate, potassium carbonate, potassium chloride, potassium iodate, potassium iodide, potassium sulfate, and/or the like. Illustrative examples of iron salts include iron ammonium citrate, iron carbonate, iron chloride, iron gluconate, iron oxide, iron phosphate, iron pyrophosphate, iron sulfate, reduced iron, and/or the like. Illustrative examples of zinc salts include zinc acetate, zinc carbonate, zinc chloride, zinc oxide, zinc sulfate, and/or the like.

Illustrative enzymes included in the silage composition may include, but are not limited to, cellulase, hemicellulase, amylase, and/or the like. The various enzymes may be included in the silage composition to hydrolyze various starches present in the silage composition, such as, for example, cellulose, hemicellulose, and/or other complex sugars. Hydrolysis of such starches may assist in the fermentation process, as described in greater detail herein.

FIG. 1 depicts a flow diagram of a representative method of preparing a silage composition, such as, for example, any of the silage compositions described herein. In various embodiments, the silage composition may be formulated in such a manner so that when consumed by a ruminant, the silage composition maximizes particular qualities in the milk produced by the ruminant, as well as an amount of milk produced by the ruminant, as described in greater detail herein.

In various embodiments, the components described herein with respect to FIG. 1 may generally be combined in any order and/or in any combination, and are not limited by the order described herein. In some embodiments, a silage composition may be prepared by providing 105 and/or processing 110 a first component and providing 115 and/or processing 120 a second component. In various embodiments, the first component may include a fatty acid composition, such as, for example, the fatty acid compositions described in greater detail herein.

Since the first component may include a fatty acid composition, it may not be combinable with other ingredients without first processing 110 the first component. For example, processing 110 the first component may ensure that the fatty acid can be combined with other ingredients, as described in greater detail herein. In some embodiments, processing 110 the first component may include, for example, dispersing the first component in water and/or heating the first component.

In some embodiments, the first component may be dispersed in an amount of water to obtain a liquid suspension. For example, the first component may include the fatty acid composition and water in a volume/volume ratio from about 1:20 to about 1:1, from about 1:15 to about 2:1, from about 1:10 to about 3:1, including about 1:20, about 1:15, about 1:10, about 1:5, about 1:3, about 1:2, about 1:1, about 2:1, about 3:1, or any value or range between any two of these values (including endpoints).

In some embodiments, the first component may be heated to obtain a melted fatty acid composition. Thus, the first component may be heated such that it reaches or exceeds a temperature that is equivalent to the melting point of the fatty acid composition or such that the first component reaches a liquid or semisolid state. One illustrative temperature may be equal to or greater than about 40° C. Another illustrative temperature may be equal to or less than about 80° C. Another illustrative temperature may be about 40° C. to about 80° C. Other illustrative temperatures may include about 40° C., about 45° C., about 50° C., about 55° C., about 60° C., about 65° C., about 70° C., about 75° C., about 80° C., or any value or range between any two of these values (including endpoints).

The second component may generally be a component that is fermentable. In some embodiments, the second component may be a green plant. In some embodiments, the second component may be at least one of a grass, a weed, a clover, alfalfa, a vetch, straw, oat, rye, sorghum, a cereal grain, hay, or maize. Those having ordinary skill in the art will recognize other second components that are fermentable without departing from the scope of the present disclosure.

Similar to the first component, the second component may be processed 120 to ensure combinability. The second component may also be processed 120 to ensure a particular fineness, such as, for example, a fineness necessary for extrusion, as described in greater detail herein. In some embodiments, the second component may be processed 120 by grinding. Grinding may provide various benefits, such as improving certain characteristics of the second component and/or the silage composition formed therefrom. For instance, even and fine particle size may improve the mixing of different ingredients. According to certain embodiments, grinding may be configured to decrease a particle size of certain components of the silage composition, for example, to increase the surface area open for enzymes in the gastrointestinal tract, which may improve the digestibility of nutrients, and/or to increase the acceptability or palatability of the silage composition.

Grinding may be performed by various grinding devices known to those having ordinary skill in the art, such as a hammer mill, a roller mill, a disk mill, a jet mill or the like. The silage composition and/or portions thereof (such as the protein component) may be ground to various sizes. Size can be measured in any number of ways, such as particle size (for instance, measured in millimeters), mesh sizes, surface areas, or the like. According to some embodiments, the second component (and/or the silage composition as a whole) may be ground to an average particle size of about 0.05 mm to about 10 mm. More particularly, the second component may be ground to produce a granular material having an average particle size of about 0.05 mm, about 0.1 mm, about 0.2 mm, about 0.5 mm, about 1 mm, about 2 mm, about 3 mm, about 4 mm, about 5 mm, about 6 mm, about 7 mm, about 8 mm, about 9 mm, about 10 mm, or any value or range between any two of these values. In some embodiments, the second component may be ground so that about 20% to 50% of the ground second component is retained by a mesh having openings with a size of about 10 mm and so that about 70% to about 90% of the ground second component is retained by a mesh having openings with a size of about 1 mm. In some embodiments, the second component (and/or the silage composition as a whole) may have a varying distribution of particle sizes based upon the ingredients. For example, in embodiments containing one or more wheat ingredients (as described in greater detail herein), the particle size may be distributed so that about 95% of the ground wheat ingredients are retained by a mesh having openings with a size of about 0.0625 mm and so that about 65% of the ground wheat ingredients are retained by a mesh having openings with a size of about 1 mm. In another example, such as embodiments containing one or more barley ingredients, the particle size may be distributed so that about 95% of the ground barley ingredients are retained by a mesh having openings with a size of about 0.0625 mm and so that about 60% of the ground barley ingredients are retained by a mesh having openings with a size of about 1 mm. The varying mesh sizes of each ingredient may be independent of mesh sizes for other ingredients.

In various embodiments, the first component and the second component may be combined 125 to obtain a mixture. Combination 125 may be completed by any method of combining, including, but not limited to, mixing, beating, blending, folding, stirring, tossing, whipping, up-lift fluidized-bed mixing, and the like. In some embodiments, combining 125 may occur after the second component has fermented, as described in greater detail herein.

In some embodiments, the ratio of the first component to the second component may be such that the fatty acid composition of the first component is present in the mixture in an amount of at least about 10% by weight of the mixture. In some embodiments, the silage composition may only contain the mixture. Thus, the fatty acid composition of the first component may also be present in the silage composition in an amount of at least about 10% by weight of the silage composition. In some embodiments, the fatty acid composition of the first component may be present in the silage composition in an amount of at least about 15% by weight of the silage composition. In other embodiments, the ratio of the first component to the second component may be such that the fatty acid composition of the first component is present in the mixture in an amount of less than about 10% by weight of the mixture. Thus, in embodiments where the silage composition only contains the mixture, the fatty acid composition of the first component may also be present in the silage composition in an amount of less than about 10% by weight of the silage composition.

In various embodiments, combining 125 the first component and the second component may be completed such that the fatty acid composition of the first component has a saturated fatty acid content of at least about 90% by weight of the fatty acid composition, at least about 91% by weight of the fatty acid composition, at least about 92% by weight of the fatty acid composition, at least about 93% by weight of the fatty acid composition, at least about 94% by weight of the fatty acid composition, at least about 95% by weight of the fatty acid composition, at least about 96% by weight of the fatty acid composition, at least about 97% by weight of the fatty acid composition, at least about 98% by weight of the fatty acid composition, at least about 99% by weight of the fatty acid composition, or any value or range between any two of these values (including endpoints).

In some embodiments, the mixture may only contain the first ingredient and the second ingredient. In other embodiments, the mixture may be combined 130 with additional ingredients. Illustrative additional ingredients may include, for example, at least one additive such as urea, anhydrous ammonia, a mineral, an acid, or an enzyme. As previously described herein, the mineral may be one or more of an ion of calcium, phosphorus, sulfur, magnesium, or a combination thereof. As also previously described herein, the acid may be one or more of formic acid, a mineral acid such as sulfuric acid and/or hydrochloric acid, propionic acid, lactic acid, and/or sodium diacetate. Those having ordinary skill in the art will recognize that other ingredients may be combined 130 with the mixture, whether or not explicitly described herein, without departing from the scope of the present disclosure.

In various embodiments, the mixture and/or various components thereof may be allowed 135 to ferment. Allowing 135 the mixture and/or various components thereof to ferment may generally include placing the mixture into a structure configured to exclude oxygen and/or provide an anaerobic environment necessary for fermentation. For example, the mixture may be placed into a silo or the like. In some embodiments, allowing 135 the mixture and/or various components thereof to ferment may include wrapping the mixture and/or various components thereof in a polymer sheet or film, as in a feed-lot bunker.

The mixture may be allowed 135 to ferment for a particular period of time. The period of time may generally be sufficient so as to allow the silage composition to become sufficiently fermented such that it can be ingested by ruminants. Illustrative periods of time may be about 1 day to about 52 weeks, about 2 weeks to about 3 weeks, about 1 month to about 5 months, about 4 months to about 6 months, about 6 months to about 12 months. In particular examples, the period of time may be about 1 day, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 16 weeks, about 26 weeks, about 52 weeks, or any other value or range between any two of these values (including endpoints). In some embodiments, the mixture may be allowed 135 to ferment until it has been consumed by one or more ruminants. In some embodiments, the mixture may be allowed 135 to ferment until it achieves a particular pH value. For example, the pH value may be such that the lactic acid that is produced as a result of fermentation stably preserves the mixture in an anaerobic environment until it is consumed. Furthermore, those having ordinary skill in the art will recognize other and additional processes that can be used to allow 135 fermentation without departing from the scope of the present disclosure.

In various embodiments, a method of increasing milk fat content in ruminants may include providing at least the silage composition as described herein to the ruminant for ingestion. In some embodiments, the silage composition may be mixed with one or more other feed ingredients before providing it to the ruminant. In particular embodiments, the silage composition may be mixed with other feed ingredients by an end user, such as a dairy farmer, feed-lot operator, and/or the like. Thus, the end user may receive the silage composition from a manufacturer, a distributor, and/or the like, may mix the silage composition with the other feed ingredients, and may provide the mixture to the ruminant. In other embodiments, the silage composition may be directly fed to a ruminant without mixing with another feed ingredient.

In various embodiments, the silage composition may be provided to the ruminant in an amount such that the ruminant receives at least about 10 grams of fatty acid per kilogram of milk produced by the ruminant each day. In other embodiments, additional fatty acid may be administered to the ruminant to ensure the ruminant receives at least about 10 grams of fatty acid per kilogram of milk produced by the ruminant each day, particularly in embodiments where the ruminant does not consume enough silage composition to receive a sufficient amount of the fatty acid. The amount of silage and/or fatty acid may be based on the previous day's milk production by the ruminant, an average day based on the previous week's milk production by the ruminant, an average day based on the previous month's milk production by the ruminant, an average production of milk by the ruminant when not provided the silage composition, and/or the like. In some embodiments, the ruminant may be provided with about 0.5 kg to about 10 kg of the silage composition each day per 30 kg of weight of the ruminant, including about 0.5 kg, about 0.75 kg, about 1 kg, about 1.5 kg, about 2 kg, about 2.5 kg, about 3 kg, about 3.5 kg, about 4 kg, about 4.5 kg, about 5 kg, about 5.5 kg, about 6 kg, about 6.5 kg, about 7 kg, about 7.5 kg, about 8 kg, about 8.5 kg, about 9 kg, about 9.5 kg, about 10 kg, or any value or range between any two of these values (including endpoints). In some embodiments, the ruminant may be provided with additional amounts of the silage composition to make up for portions of the silage composition that are not consumed by the ruminant, such as amounts that are spilled by the ruminant when consuming the silage composition, amounts that are consumed by other animals, and/or the like.

In some embodiments, providing the silage composition to the ruminant for the ruminant to consume may result in increased milk production and/or an increase in fat content of the milk produced. These increases may generally be relative to a similar ruminant that does not receive the silage composition, an average of similar ruminants not receiving the silage composition, an average of the milk production quantity and fat content of the same ruminant when not provided the silage composition, and/or the like. In particular embodiments, the milk production may increase by an amount of about 1% to about 10%, including about 1%, about 2%, about 3%, about 4%, about 5%, about 6%, about 7%, about 8%, about 9%, about 10%, or any value or range between any two of these values. In particular embodiments, the milk fat content may increase by an amount of about 10% to about 15%, including about 10%, about 11%, about 12%, about 13%, about 14%, about 15%, or any value or range between any two of these values.

### Examples

### Example 1: Making a Silage Composition

A silage composition to be fed to ruminants is made using a process of combining a first component and a second component. The first component will have a fatty acid composition. The second component will be a fermentable material.

The first component is combined in an amount that is about 20% by weight of the silage composition. The fatty acid composition includes about 99% by weight of a palmitic acid composition and about 1% by weight unsaturated trans fatty acids. The silage composition also includes 75% by weight of the second component. The second component will be a mixture of grass, weeds, clovers, alfalfa plants, vetches, and cereal grains. In addition, various additives totaling about 5% by weight of the silage composition will also be combined with the first component and the second component. Such additives include urea, various calcium salts, various magnesium salts, formic acid, propionic acid, and sulfuric acid.

Prior to combining, the first component will be melted to a temperature of about 63° C. to ensure the fatty acid composition is sufficiently melted to be combined with the remaining ingredients of the silage composition. In addition, the second component, prior to combining, will be ground using a hammer mill such that the second component has an average particle size of 10 mm.

Once the first component, the second component, and the additives are combined, they will be placed in a silo and allowed to ferment for 3 months. The silo will provide a generally anaerobic environment sufficient for fermentation.

### Example 2: Making a Silage Composition

Similar to the process described above with respect to Example 1, a silage composition to be fed to ruminants is made using a process of combining a first component and a second component. The first component will have a fatty acid composition. The second component will be a fermentable material.

The first component is combined in an amount that is about 5% by weight of the silage composition. The fatty acid composition includes about 100% by weight of a palmitic acid composition. The silage composition also includes about 90% by weight of the second component. The second component will be a mixture of grass, weeds, and hay. In addition, various additives totaling about 5% by weight of the silage composition will also be combined with the first component and the second component. Such additives include anhydrous ammonia, sodium sulfate, potassium carbonate, hydrochloric acid, and sulfuric acid.

Prior to combining, the first component will be melted to a temperature of about 63° C. to ensure the fatty acid composition is sufficiently melted to be combined with the remaining ingredients of the silage composition. In addition, the second component, prior to combining, will be ground using a hammer mill such that the second component has an average particle size of 10 mm.

Once the first component, the second component, and the additives are combined, they will be placed in a silo and allowed to ferment for at least 3 months. The silo will provide a generally anaerobic environment sufficient for fermentation.

### Example 3: Feeding a Dairy Cow

A silage composition as described above in Example 1 is provided to a cow for consumption each day. The cow is provided with an amount of the silage composition that ensures that an average dairy cow weighing about 635 kg will consume about 5 kg of the silage composition per 30 kg of her weight. Such an amount corresponds to at least about 10 grams of free palmitic acid for every kilogram of milk that she produces that day.

The cow has a normal (untreated) average daily production of 30 kg milk. The cow is provided with the silage described above to increase the milk fat and the quantity of the milk produced. The cow is fed this silage for 30 days. At the end of the 30 day period, she is producing 10% more milk than she did previously, and the milk that she produces contains 10% more milk fat content than the milk she produced previously.

### Example 4: Providing to a Large Group of Cows

A silage composition as described in Example 1 is provided to a large group of cows on a commercial dairy farm to confirm its effectiveness. A group of 200 dairy cows from the commercial dairy farm are selected at random to provide a wide variety of variation in various characteristics, such as breed, weight, age of the cow, and the like. The 200 cows are divided into two groups: a sample cow group of 100 cows and a control cow group of 100 cows. Each day, the sample cow group is fed the silage composition ad libitum. The control cow group is fed a standard, commercially-available TMR feed ad libitum. The 200 cows are monitored for the amount of feed and/or silage consumed, changes in weight, an amount of milk the cow produces each day, and the composition of the milk produced by the cow each day. Monitoring continues for a period of 30 days. A comparison of the two groups of cows over this period of time shows a statistically significant improvement for the group that consumed the silage composition over the control group that did not receive the silage composition.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A silage composition comprising:
a fatty acid component comprising at least about 70% saturated fatty acid by weight; and
a fermented component;
wherein the fatty acid component is present in the silage composition in an amount of at least about 10% by weight of the silage composition.

2. The silage composition of claim 1, wherein the silage composition consists essentially of the fatty acid component and the fermented component.

3. The silage composition of claim 1, wherein the silage composition consists of the fatty acid component and the fermented component.

4. The silage composition of claim 1, wherein the fermented component comprises a green plant.

5. The silage composition of claim 1, wherein the fermented component comprises at least one of a grass, a weed, a clover, alfalfa, a vetch, straw, oat, rye, sorghum, a cereal grain, hay, or maize.

6. The silage composition of claim 1, wherein the saturated fatty acid comprises a palmitic acid compound.

7. The silage composition of claim 6, wherein the palmitic acid compound comprises free palmitic acid.

8. The silage composition of claim 6, wherein the palmitic acid compound comprises a palmitic acid derivative selected from a palmitic acid ester, a palmitic acid phosphonate, a palmitic acid amide, a palmitic acid salt, a palmitic acid carbonate, a palmitic acid carbamate, a palmitic acid imide, a palmitic acid anhydride, or a combination thereof.

9. (canceled)

10. The silage composition of claim 1, wherein the saturated fatty acid comprises a palmitic acid compound in an amount of at least about 80% by weight of the fatty acid component.

11. The silage composition of claim 1, wherein the saturated fatty acid comprises a palmitic acid compound in an amount of at least about 90% by weight of the fatty acid component.

12.-17. (canceled)

18. The silage composition of claim 1, wherein the fatty acid component is substantially free of unsaturated trans fatty acids.

19. (canceled)

20. The silage composition of claim 1, wherein the saturated fatty acid is present in the fatty acid component in an amount of at least about 98% by weight of the fatty acid component.

21.-22. (canceled)

23. The silage composition of claim 1, wherein the fatty acid component has a melting point equal to or greater than about 40° C.

24. (canceled)

25. The silage composition of claim 1 further comprising at least one additive selected from urea, anhydrous ammonia, a mineral, an acid, and an enzyme.

26. The silage composition of claim 1, further comprising at least one mineral comprising an ion of calcium, phosphorus, sulfur, magnesium, or a combination thereof.

27.-28. (canceled)

29. The silage composition of claim 1, further comprising at least one acid selected from formic acid, a mineral acid, propionic acid, lactic acid, and sodium diacetate.

30. (canceled)

31. The silage composition of claim 1, further comprising at least one enzyme selected from cellulase, hemicellulase, and amylase.

32. A method of preparing a silage composition for ruminants, the method comprising:
combining a first component comprising a fatty acid composition and a second component to form a mixture such that the fatty acid is present in the mixture in an amount of at least about 10% by weight of the mixture; and
allowing the mixture to ferment.

33. (canceled)

34. The method of claim 32, further comprising grinding the second component prior to combining with the first component.

35. The method of claim 32, further comprising dispersing the first component in water to obtain a liquid suspension prior to combining with the second component.

36.-37. (canceled)

38. The method of claim 32, wherein combining the first component comprises combining, with the second component, a fatty acid composition comprising saturated fatty acid in an amount of at least about 95% by weight of the fatty acid composition.

39. The method of claim 32, wherein combining the first component comprises combining, with the second component, a fatty acid composition comprising saturated fatty acid in an amount of at least about 98% by weight of the fatty acid composition.

40. (canceled)

41. The method of claim 32, wherein combining the second component comprises combining, with the first component, at least one of a grass, a weed, a clover, alfalfa, a vetch, straw, oat, rye, sorghum, a cereal grain, hay, or maize.

42. The method of claim 32, further comprising combining at least one additive with the mixture, wherein the additive is selected from at least one of urea, anhydrous ammonia, a mineral, an acid, and an enzyme.

43. The method of claim 32, further comprising combining at least one mineral with the mixture, wherein the mineral comprises an ion of calcium, phosphorus, sulfur, magnesium, or a combination thereof.

44. The method of claim 32, further comprising combining at least one acid with the mixture, wherein the acid is selected from at least one of formic acid, a mineral acid, propionic acid, lactic acid, and sodium diacetate.

45. (canceled)

46. A method of increasing milk fat content in ruminants, the method comprising:
providing a silage composition to a ruminant for ingestion, wherein the silage composition comprises:
a fatty acid component comprising at least about 70% saturated fatty acid by weight, wherein the fatty acid component is present in the silage composition in an amount of at least about 10% by weight of the silage composition, and
a fermented component.

47. The method of claim 46, wherein providing the silage composition to the ruminant comprises providing about 0.5 kg to about 1.5 kg of silage composition to the ruminant daily.

48. The method of claim 46, wherein providing the silage composition to the ruminant comprises providing the silage composition to the ruminant at an amount such that the ruminant receives at least about 10 grams of fatty acid per kilogram of milk produced by the ruminant per day.

49. The method of claim 46, wherein providing the silage composition to the ruminant results in at least one of an increase in production of milk by the ruminant and an increase in a fat content in the milk produced by the ruminant, relative to a similar ruminant not provided the silage composition.

50. The method of claim 46, wherein providing the silage composition to the ruminant results in at least one of an at least about 1% increase in production of milk by the ruminant and an at least about 10% increase in a fat content in the milk produced by the ruminant, relative to a similar ruminant not provided the silage composition.

51. A silage composition comprising:
a fatty acid component comprising at least about 70% saturated fatty acid by weight; and
a fermented component;
wherein the fatty acid component is present in the silage composition in an amount of less than about 10% by weight of the silage composition.

52. A method of preparing a silage composition for ruminants, the method comprising:
combining a first component comprising a fatty acid composition and a second component to form a mixture such that the fatty acid is present in the mixture in an amount of less than about 10% by weight of the mixture; and
allowing the mixture to ferment.

53. A method of increasing milk fat content in ruminants, the method comprising:
providing a silage composition to a ruminant for ingestion, wherein the silage composition comprises:
a fatty acid component comprising at least about 70% saturated fatty acid by weight, wherein the fatty acid component is present in the silage composition in an amount of less than about 10% by weight of the silage composition, and
a fermented component.
